(19) United States
(12) Patent Application Publication
(10) Pub. No.: US 2026/0053835 A1
(43) Pub. Date: Feb. 26, 2026

(54) Anti-Aging Formulation Comprising Human Milk Oligosaccharides, Ellagic Acid, and Probiotics to Enhance Urolithin A Production

(71) Applicants: Zhong Zhang, Marietta, GA (US); Jing Guo, Roswell, GA (US)

(72) Inventors: Zhong Zhang, Marietta, GA (US); Jing Guo, Roswell, GA (US)

(73) Assignee: Mellitas Health Foods, LLC, Ithaca, NY (US)

(21) Appl. No.: 18/810,913

(22) Filed: Aug. 21, 2024

(51) Int. Cl. A61K 31/702; A61K 31/366; A61K 35/00; A61K 35/745; A61K 35/747

(52) U.S. Cl. CPC A61K 31/702; A61K 31/366; A61K 35/745; A61K 35/747; A61K 2035/115

(57) ABSTRACT

This invention provides a comprehensive approach to anti-aging through the enhancement of Urolithin A production by targeting specific gut bacteria with a novel formulation. By combining 2-fucosyllactose, ellagic acid, HMOs, and probiotics, the formulation not only boosts Urolithin A levels but also promotes a diverse and healthy gut microbiome, leading to improved mitochondrial function and overall health.

### BACKGROUND OF THE INVENTION

Aging is a biological process characterized by the decline in cellular and mitochondrial function, contributing to various age-related diseases and reduced vitality. Urolithin A, a metabolite derived from gut microbiota, has been shown to improve mitochondrial function, promote autophagy, and exhibit anti-aging effects. However, not all individuals have the gut microbiota capable of producing Urolithin A from its precursor, which is dependent on the presence of specific bacteria working together such as Gordonibacter species and Enterocloster citroniae. [1]

2-Fucosyllactose (2-FL) is a human milk oligosaccharide (HMO) known for its prebiotic properties, particularly in promoting the growth of beneficial gut bacteria. [2] Ellagic acid, a polyphenol found in pomegranate, is the precursor for Urolithin A. [3] The present invention demonstrates that the administration of 2-FL can significantly increase the population of Gordonibacter species and Enterocloster citroniae in the gut, thereby enhancing Urolithin A production in the human gut.

### SUMMARY OF THE INVENTION

The invention provides a novel formulation for anti-aging, comprising 2-fucosyllactose, ellagic acid from pomegranate, other human milk oligosaccharides, and probiotics. The formulation is designed to enhance the growth and activity of Gordonibacter species and Enterocloster citroniae in the gut, thereby boosting the endogenous production of Urolithin A. Our data shows that the administration of 2-fucosyllactose for 23 days increases the presence of Gordonibacter from undetectable levels to 0.0019% and Enterocloster citroniae from undetectable levels to 0.0039%.

### DETAILED DESCRIPTION OF THE INVENTION

Composition: The anti-aging formulation comprises the following components, mentioned in 2-Fucosyllactose (2-FL), Ellagic acid or Ellagitannins, human milk oligosaccharides such as Lacto-N-tetraose (LNT), Lacto-N-neotetraose (LNnT), 6′-Sialyllactose (6′SL), and 3′-Sialyllactose (3′SL), and probiotics such as bifidobacterium and lactobacillus.

2-Fucosyllactose (2-FL): Included at a concentration effective to selectively promote the growth of Gordonibacter species and Enterocloster citroniae.

Ellagic Acid: Sourced from pomegranate extract, serving as the precursor for Urolithin A production in the human gut.

Human Milk Oligosaccharides (HMOs): Additional HMOs such as lacto-N-tetraose (LNT) or 3′-sialyllactose (3′-SL) to further enhance the prebiotic effect and support a diverse gut microbiota.

Probiotics: Specific strains of Bifidobacterium and Lactobacillus species are included to seed and diversify the gut microbiome.

Optional Prebiotic Fibers: Additional prebiotic fibers like inulin or galactooligosaccharides (GOS) may be added to further support the growth of beneficial gut bacteria.

Mechanism of Action 1: The formulation works through the following mechanisms from Enhancement of Urolithin A Production: 2-FL and ellagic acid synergistically promote the growth and activity of Gordonibacter species and Enterocloster citroniae, which work together to convert ellagic acid into Urolithin A in the human gut.

Mechanism of Action 2: Diversification of Gut Microbiota: The inclusion of additional HMOs and probiotics supports a diverse and balanced gut microbiome, which is essential for optimal gut health and overall well-being.

Mechanism of Action 3: Support of Mitochondrial Health: By increasing the endogenous production of Urolithin A, the formulation supports mitochondrial function, autophagy, and cellular regeneration, contributing to anti-aging effects.

Method of Use: The formulation is intended for daily use as a dietary supplement. It can be administered in various forms, such as capsules, powders, or beverages. The recommended dosage is adjusted to ensure optimal delivery of 2-FL, ellagic acid, HMOs, and probiotics, tailored to the individual's needs for promoting gut health and enhancing Urolithin A production.

Data from experimental study demonstrates that the administration of the 2-fucosyllactose increases the population of Gordonibacter species in the gut from undetectable levels to 0.0019% and Enterocloster citroniae from undetectable levels to 0.0039%. These findings indicate that the formulation effectively enhances the gut microbiota's ability to produce Urolithin A.

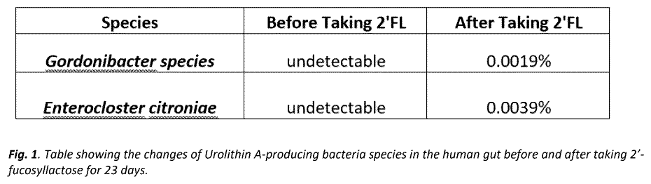

FIG. 1 is a Table showing the changes of Urolithin A-producing bacteria species in the human gut before and after taking 2'-fucosyllactose for 23 days.

### EXAMPLES

### Example 1

A daily supplement comprising 4000 mg of 2-fucosyllactose. This formulation is designed to promote the growth of Urolithin A-producing bacteria and support a diverse gut microbiome.

### Example 2

A daily supplement comprising 200 mg of 2-fucosyllactose, 200 mg of ellagic acid from pomegranate extract, 300 mg of lacto-N-tetraose, and 10\hat{ }9 CFU each of Bifidobacterium lactis and Lactobacillus rhamnosus. This formulation is designed to promote the growth of Urolithin A-producing bacteria and support a diverse gut microbiome.

### Example 3

A powder formulation containing 2-fucosyllactose (500 mg), ellagic acid (250-1000 mg), 6′-sialyllactose (50 mg), 3′-sialyllactose (30 mg), and a prebiotic fiber blend (inulin, 300 mg) with probiotics (Bifidobacterium breve and Lactobacillus plantarum, 10\hat{ }9 CFU each) to enhance gut health and Urolithin A production.

1. A formulation for anti-aging comprising 2-fucosyllactose, ellagic acid, or at least one additional human milk oligosaccharide to enhance the production of Urolithin A in the human gut by promoting the growth of Gordonibacter species and Enterocloster citroniae.

2. The formulation of claim 1, where 2-fucosyllactose, simultaneously promotes the growth of Gordonibacter species and Enterocloster citroniae.

3. The formulation of claim 1, further comprising probiotics selected from the group consisting of Bifidobacterium and Lactobacillus species to diversify the gut microbiota.

4. The formulation of claim 1, further comprising additional prebiotic fibers to support the growth of beneficial gut bacteria.

5. The formulation of claim 1, wherein the ellagic acid is sourced from pomegranate extract or other natural sources or synthetic sources.

6. The formulation of claim 1, wherein a method of enhancing mitochondrial function and providing anti-aging benefits, comprising administering to a subject a formulation containing 2-fucosyllactose, ellagic acid, additional HMOs, and probiotics to increase the production of Urolithin A in the gut.
